(19) United States
(12) Patent Application Publication
(10) Pub. No.: US 2022/0017444 A1
(43) Pub. Date: Jan. 20, 2022

(54) BENZHYDRYLATED AROMATIC SURFACTANTS

(71) Applicant: INDORAMA VENTURES OXIDES LLC, The Woodlands, TX (US)

(72) Inventors: Alan J. STERN, The Woodlands, TX (US); Matthew T. MEREDITH, The Woodlands, TX (US)

(21) Appl. No.: 17/296,098

(22) PCT Filed: Nov. 8, 2019
(86) PCT No.: PCT/US2019/060525

§ 371 Date: May 21, 2021

Related U.S. Application Data
(60) Provisional application No. 62/780,979, filed on Dec. 18, 2018.

(51) Int. Cl. C07C 43/23; C07C 41/03; A01N 25/04; A01N 25/14; A61K 8/34; A61Q 19/00

(52) U.S. Cl. CPC C07C 43/23; C07C 41/03; A01N 25/04; A61K 2800/10; A61K 8/34; A61Q 19/00; A01N 25/14

(57) ABSTRACT

The present disclosure provides an alkoxylate compound containing aromatic groups in the hydrophobe allowing the compound to exhibit unique functionality, high performance and low cost, but without the toxicity and/or skin and eye irritation problems associated with conventional alkylphenol and mono-, di- and tristyrylphenol compounds.

### CROSS REFERENCE TO RELATED APPLICATIONS

The present application claims priority to U.S. Provisional Patent Application Ser. No. 62/780,979, filed Dec. 18, 2018, the entire contents of which is hereby expressly incorporated herein by reference.

### FIELD

The present disclosure generally relates to aromatic-based alkoxylate compounds derived from benzhydrol, methods for preparing such compounds and their use in a variety of applications including, but not limited to, cleaning, fabric treatment, hair conditioning, fuel additive, oil field, agricultural, personal care and antimicrobial formulations, metal-working fluids and in the manufacture of polyurethane foam.

### BACKGROUND

Surfactants and dispersants containing aromatic groups in the hydrophobe are important in many different applications due to their unique functionality, high performance and low cost. For example, it is known phenols may be reacted with olefins (e.g. 1-nonene or 1-octene) to produce alkylphenol hydrophobes while styrene may be reacted with phenol to produce mono-, di- and tristyrylphenols. These aromatic-based hydrophobes may then be ethoxylated to produce surfactants and employed in a variety of applications including cosmetics, detergents, cleaners, toiletries, oil slick dispersants, deinking surfactants, metal treatment formulations, textile treatments, emulsion formulations and emulsion polymerizations.

However, both of these surfactant classes based on aromatic hydrophobes exhibit undesirable properties. In particular, recent studies have suggested that these conventional ethoxylates experience delayed or deficient biodegradation and can generate biodegradation products, such as alkylphenols and tristyrylphenol. Alkylphenols have been found to be endocrine disruptors while alkylphenols and tristyrylphenol are known to be persistent environmental pollutants. In addition, styrene used in the production of mono-, di- and tristyrylphenol is known to also exhibit undesirable toxicity properties while being highly flammable. Thus, the storage and handling of this compound in an industrial setting poses significant safety risks and is becoming highly disfavored.

Considering the hazardous and potential ecological effects due to manufacturing, processing and distribution of ethoxylates derived from the above described aromatic-based hydrophobes, there remains a significant need to identify, develop and employ alternatives to these conventional hydrophobes and the alkoxylates derived therefrom which are not only highly effective in altering surface properties at an interface but also do not possess the disadvantageous characteristics described above.

### SUMMARY

According to one embodiment, the present disclosure provides a benzhydrylated alkoxylate comprising a compound having the formula (1)

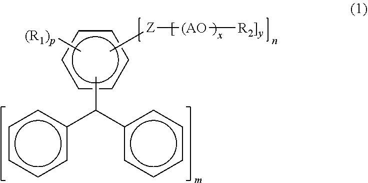

where: each R₁ is independently hydrogen, an alkyl group, a phenyl group, a group having a structure (2)

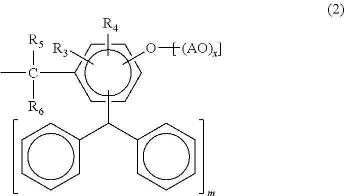

or two R₁'s in combination form a C₄H₄ di-radical having a structure (3)

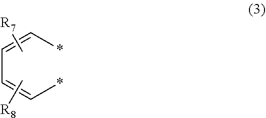

where * represents a binding site to the benzene ring;
each (AO) unit is independently an alkoxy group;
p is an integer of 1 to 4;
each R₂ is independently hydrogen, SO₃M, COOM or PO₃M₂;
M is hydrogen, a water-soluble cation, a monovalent metal or a polyvalent metal cation;
Z is sulfur, oxygen or nitrogen with the proviso that when Z is sulfur, y is 1 and n is 1 and when Z is oxygen, y is 1 and n is 1 or 2 and when Z is nitrogen, y is 2 and n is 1;
x is an integer of 1 to 100;
m is an integer of 1 to 3;
R₃ and R₄ are independently hydrogen, an alkyl group or a phenyl group;
R₅ and R₆ are independently hydrogen, methyl or a phenyl group; and
R₇ and R₈ are independently hydrogen or an alkyl group.

In a further embodiment, there is provided a method of making the benzhydrylated alkoxylate compound of formula (1) by: reacting benzhydrol with an aromatic compound selected from phenol optionally substituted with at least one alkyl group, phenyl group or combination thereof, a bisphenol, a benzenediol optionally substituted with at least one alkyl group, naphthol, thiophenol and aniline optionally substituted with at least one alkyl group to form a benzhydrylated hydrophobe and (ii) alkoxylating the benzhydrylated hydrophobe with an alkylene oxide to form the benzyhydrylated alkoxylate of formula (1). In still further embodiments, the benzhydrylated alkoxylate may optionally be further reacted with an acid moiety and neutralized with a source of alkali metal, alkaline earth metal, amine or ammonia.

In still another embodiment, there is provided uses of the benzhydrylated alkoxylate compounds of the present disclosure, such as in performance chemical formulations or personal care formulations and in the manufacture of polyurethane foam.

### BRIEF DESCRIPTION OF THE DRAWINGS

FIGS. 1 and 2 depict the surface tension and critical micelle concentration of the benzhydrylated alkoxylates of the present disclosure.

### DETAILED DESCRIPTION

The following terms shall have the following meanings.

If appearing herein, the term “comprising” and derivatives thereof are not intended to exclude the presence of any additional component, step or procedure, whether or not the same is disclosed herein. In order to avoid any doubt, all compositions claimed herein through use of the term “comprising” may include any additional additive, adjuvant, or compound, unless stated to the contrary. In contrast, the term, “consisting essentially of” if appearing herein, excludes from the scope of any succeeding recitation any other component, step or procedure, except those that are not essential to operability and the term “consisting of”, if used, excludes any component, step or procedure not specifically delineated or listed. The term “or”, unless stated otherwise, refers to the listed members individually as well as in any combination.

The articles “a” and “an” are used herein to refer to one or to more than one (i.e. to at least one) of the grammatical objects of the article. By way of example, “an alkylene oxide” means one alkylene oxide or more than one alkylene oxide. The phrases “in one embodiment”, “according to one embodiment” and the like generally mean the particular feature, structure, or characteristic following the phrase is included in at least one embodiment of the present disclosure, and may be included in more than one embodiment of the present disclosure. Importantly, such phrases do not necessarily refer to the same embodiment. If the specification states a component or feature “may”, “can”, “could”, or “might” be included or have a characteristic, that particular component or feature is not required to be included or have the characteristic.

The terms “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the present disclosure.

The term “about” as used herein can allow for a degree of variability in a value or range, for example, it may be within 10%, within 5%, or within 1% of a stated value or of a stated limit of a range.

Values expressed in a range format should be interpreted in a flexible manner to include not only the numerical values explicitly recited as the limits of the range, but to also include all of the individual numerical values or sub-ranges encompassed within that range as if each numerical value and sub-range is explicitly recited. For example, a range from 1 to 6, should be considered to have specifically disclosed sub-ranges, such as from 1 to 3, from 2 to 4, from 3 to 6, etc., as well as individual numbers within that range, for example, 1, 2, 3, 4, 5, and 6. This applies regardless of the breadth of the range

The term “alkyl” is inclusive of both straight chain and branched chain groups and of cyclic groups. Straight chain and branched chain groups may have up to 20 carbon atoms unless otherwise specified. Cyclic groups can be monocyclic or polycyclic, and in some embodiments, can have from 3 to 10 carbon atoms.

The term “alkylphenol” means a phenol group having one or more alkyl substituents.

The term “alkoxy” means a straight or branched chain hydrocarbon ether group of 10 or less carbon atoms, including methoxy, ethoxy, 2-propoxy, propoxy, butoxy, 3-pentoxy and the like.

The term “alkoxylate” includes pure substances as well as mixtures which are obtained using different alkylene oxides.

The term “aromatic” refers to compounds that have unsaturated cyclic hydrocarbons containing one or more rings.

The term “bisphenol” refers to a polyhydric polyphenol having two phenylene groups that each includes six-carbon rings and a hydroxyl group attached to a carbon atom of the ring, wherein the rings of the two phenylene groups do not share any atoms in common.

The term “hydroxyl” or “hydroxy” as used herein is represented by the formula —OH.

The term “substantially free” means, when used with reference to the substantial absence of a component in a composition, that such a component is not present, or if at all, as an incidental impurity or by-product. In other words, the component does not affect the properties of the composition. In some embodiments, substantially free may be less than 2 wt. %, less than 1 wt. %, less than 0.5 wt. %, or less than 0.1 wt. % or less than 0.05 wt. % or even less than 0.01 wt. %, based on the total weight of the composition or that no amount of that particular component is present in the respective composition

The term “performance chemicals formulations” refers to non-personal care formulations that serve a broad variety of applications, and include non-limiting formulations such as, adhesives, agricultural, biocides, coatings, electronics, household-industrial-institutional (HI&I), inks, membranes, metal working, paper, paints, plastics, printing, plasters, oil field, polyurethane, textile and wood-care formulations.

The term “personal care formulation” refers to such illustrative non-limiting formulations as skin, sun, oil, hair, cosmetic, and preservative formulations, including those to alter the color and appearance of the skin. Potential personal care formulations include, but are not limited to, polymers for increased flexibility in styling, durable styling, and increased humidity resistance for hair, skin, and color cosmetics, sun care water-proof/resistance, wear-resistance, and thermal protecting/enhancing formulations.

The present disclosure provides novel alkoxylate compounds containing aromatic groups in the hydrophobe which allows the compounds to exhibit the unique functionality, high performance and low cost of alkylphenol ethoxylates and mono-, di- and tristyrylphenol ethoxylates, but without the toxicity, flammability and/or environmental persistence problems associated with these conventional compounds. In particular, Applicants have surprisingly found alkoxylate compounds having a high density of aromatic groups can be produced by attaching additional aromatic groups onto a phenolic or aniline ring prior to alkoxylation. Because the alkoxylate compounds of the present disclosure comprise a high density or aromatic groups, they may exhibit unique surfactancy properties that may be at least comparable, and in some embodiments even improved, to those for conventional aromatic-based alkoxylates.

According to one embodiment, the present disclosure provides a benzyhydrylated alkoxylate comprising a compound having a general formula (1)

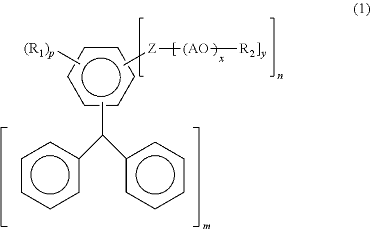

where: each R₁ is independently hydrogen, an alkyl group, a phenyl group, a group having a structure (2)

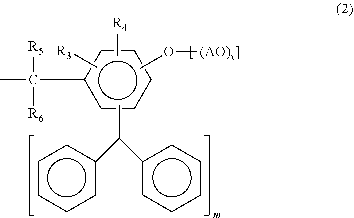

or two R₁'s in combination form a C₄H₄ di-radical having a structure (3)

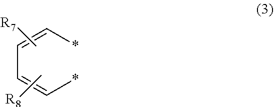

where * represents a binding site to the benzene ring;
each (AO) unit is independently an alkoxy group;
p is an integer of 1 to 4;
each R₂ is independently hydrogen, SO₃M, COOM or PO₃M₂;
M is hydrogen, a water-soluble cation, a monovalent metal or a polyvalent metal cation;
Z is sulfur, oxygen or nitrogen with the proviso that when Z is sulfur, y is 1 and n is 1 and when Z is oxygen, y is 1 and n is 1 or 2 and when Z is nitrogen, y is 2 and n is 1;
x is an integer of 1 to 100;
m is an integer of 1 to 3;
R₃ and R₄ are independently hydrogen, an alkyl group or a phenyl group;
R₅ and R₆ are independently hydrogen, methyl or a phenyl group; and
R₇ and R₈ are independently hydrogen or an alkyl group. In some embodiments, M may be hydrogen, sodium, potassium, magnesium, or calcium.

The benzhydrylated alkoxylate compound of formula (1) may generally be prepared by (i) reacting benzhydrol with an aromatic compound selected from phenol optionally substituted with at least one alkyl group, phenyl group or combination thereof, a bisphenol, a benzenediol optionally substituted with at least one alkyl group, naphthol and aniline optionally substituted with at least one alkyl group to form a benzhydrylated hydrophobe (ii) alkoxylating the benzhydrylated hydrophobe with an alkylene oxide to form the benzyhydrylated alkoxylate of formula (1) and optionally (iii) converting the benzhydrylated alkoxylate of formula (1) to a sulfonate, carboxylate or phosphate by conventional techniques, such as further reaction with an acidic moiety and optional neutralization with a source of alkali metal or alkaline earth metal.

Thus, according to one embodiment, the benzhydrylated alkoxylate compound of formula (1) may be derived from benzhydrol and phenol and comprises a compound having a general formula (4)

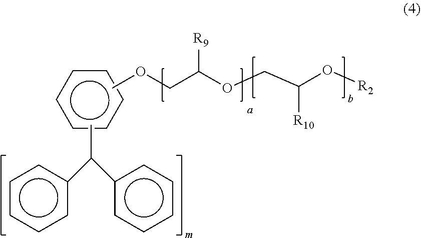

where each R₉ and R₁₀ are independently hydrogen, methyl or ethyl; R₂ is hydrogen, SO₃M, COOM or PO₃M₂; M is hydrogen, a water-soluble cation, a monovalent metal or a polyvalent metal cation; m is an integer of 1 to 3; a is an integer of 0 to 100; b is an integer of 0 to 100 and wherein a+b=1 to 100. In some embodiments, M may be hydrogen, sodium, potassium, magnesium, or calcium.

According to one embodiment, in the compound of formula (4), m is 1. In another embodiment, in the compound of formula (4), m is 2 or 3. In a further embodiment, in the compound of formula (4), R₂ is hydrogen. In still another embodiment, in the compound of formula (4), R₂ is SO₃M or PO₃M where M is hydrogen, sodium potassium, magnesium or calcium. In still another embodiment, in the compound of formula (4), a is 0 to 50, b is 0 to 50 and a+b=1 to 50. In still other embodiments, in the compound of formula (4), a is 0 to 20, b is 0 to 20 and a+b=1 to 20, while in further embodiments, a is 0 to 10, b is 0 to 10 and a+b=1 to 10. In still yet another embodiment, in the compound of formula (4), R₉ is hydrogen and R₁₀ is methyl or ethyl, in some embodiments methyl, and a is 1 to 60 and b is 0 to 20. In still other embodiments, R₉ and R₁₀ are chosen such that the a groups and the b groups are random or block groups of ethylene oxide, propylene oxide or butylene oxide. In other embodiments, the oxyalkylene a and b groups may also be mixed randomly or in blocks. In one particular embodiment, in the compound of formula (4), each R₉ and R₁₀ independently is hydrogen or methyl and the oxyalkylene groups a and b are arranged in block or random groups of ethylene oxide and propylene oxide. In yet a further embodiment, R₉ is hydrogen, a is 1 to 100, or 1 to 60, or 1 to 30, or 1 to 25, or 1 to 20, or 1 to 15, or 1 to 10 and b is 0.

According to another embodiment, the benzhydrylated alkoxylate compound of formula (1) may be derived from benzhydrol and phenol substituted with an alkyl group or phenyl group and comprises a compound having a general formula (5)

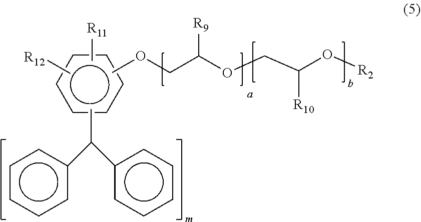

where R₁₁ and R₁₂ are independently an alkyl group or a phenyl group and R₂, m, R₉, R₁₀, a and b are defined as above.

In one particular embodiment, in the compound of formula (5), R₁₁ and R₁₂ are independently a C₁-C₁₈ alkyl group or a phenyl group, or a C₁-C₁₂ alkyl group or a phenyl group, or C₁-C₆ alkyl group or a phenyl group or a C₁-C₃ alkyl group or a phenyl group. In yet another embodiment, R₁₁ and R₁₂ are independently a C₁-C₁₆ alkyl group or a C₁-C₁₀ alkyl group or a C₁-C₄ alkyl group or a methyl group.

According to another embodiment, the benzhydrylated alkoxylate compound of formula (1) may be derived from benzhydrol and a benzenediol which may be optionally substituted with an alkyl group and comprises a compound having a general formula (6)

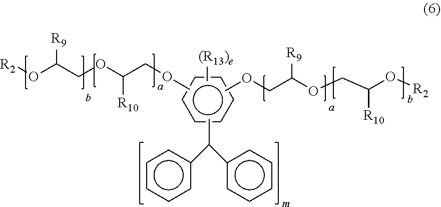

where each R₁₃ is independently an alkyl group, e is an integer of 0 to 3 and R₂, m, R₉, R₁₀, a and b are defined as above.

In one particular embodiment, in the compound of formula (6), e is 0. In another embodiment, in the compound of formula (6), e is 1 to 3 and each R₁₃ is independently a C₁-C₁₂ alkyl group, or a C₁-C₁₀ alkyl group, or a C₁-C₆ alkyl group or a C₁-C₄ alkyl group or a methyl group.

According to yet another embodiment, the benzhydrylated alkoxylate compound of formula (1) may be derived from benzhydrol and bisphenol optionally substituted with an alkyl group or phenyl group and comprises a compound having a general formula (7)

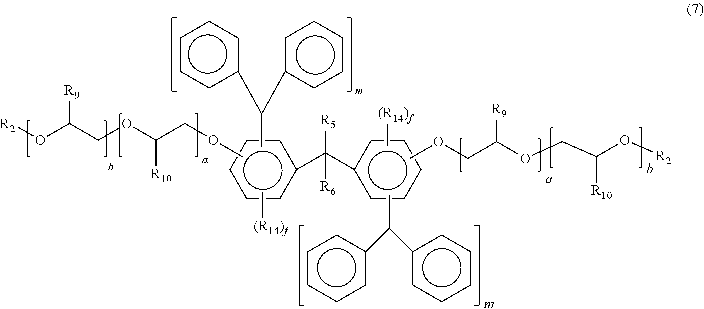

where each R₁₄ is independently a C₁-C₃ alkyl group or a phenyl group, f is 0 or 1 and R₂, m, R₉, R₁₀, a and b are defined as above.

In one embodiment, in the compound of formula (7), f is 0. In another embodiment, in the compound of formula (7), f is 1 and R₁₄ is methyl, isopropyl or phenyl.

According to another embodiment, the benzhydrylated alkoxylate compound of formula (1) may be derived from benzhydrol and naphthol and comprises a compound having a general formula (8)

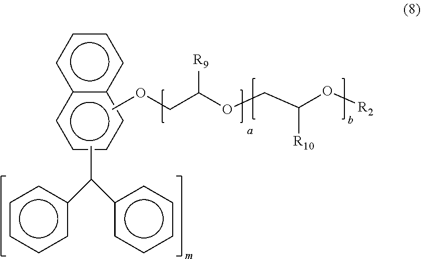

where R₂, m, R₉, R₁₀, a and b are defined as above.

In still yet another embodiment, the benzhydrylated alkoxylate compound of formula (1) may be derived from benzhydrol and aniline optionally substituted with an alkyl group and comprises a compound having a general formula (9)

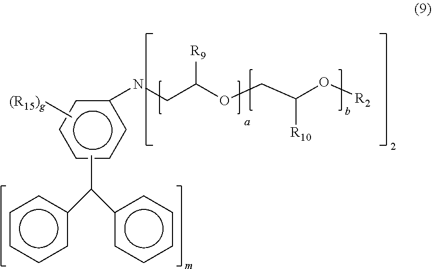

where each R₁₅ is independently an alkyl group, g is an integer of 0 to 4 and R₂, m, R₉, R₁₀, a and b are defined as above.

In one embodiment, in the compound of formula (9), g is 0. In another embodiment, in the compound of formula (9) g is 1 to 3 and each R₁₅ is independently a C₁-C₁₈ alkyl group, or a C₁-C₁₂ alkyl group, or C₁-C₆ alkyl group, or a C₁-C₃ alkyl group or a methyl group.

The benzhydrylated alkoxylate compounds of the present disclosure may generally be prepared by reacting benzhydrol with a substituted or unsubstituted aromatic compound such as an aromatic compound selected from phenol optionally substituted with an alkyl group, a phenyl group or combination thereof, a bisphenol, a benzenediol optionally substituted with an alkyl group, naphthol, and aniline optionally substituted with at an alkyl group in the presence of an acid catalyst to form a benzhydrylated hydrophobe. The benzhydrylated hydrophobe may then be directly alkoxylated using oxides such as ethylene oxide, propylene oxide, butylene oxide and mixtures thereof. If desired, the benzhydrylated alkoxylate that is produced above may be converted to a sulfonate, carboxylate or phosphate by further reaction with an acidic moiety or sodium chloroacetate, and then optionally neutralized with a source of alkali metal, alkaline earth metal, amine or ammonia.

The alkoxylation step may be catalyzed by an alkoxylation catalyst including those well known to those skilled in the art. Examples include, but are not limited to, strong bases, such as alkali metal hydroxides and alkaline earth metal hydroxides, Brønsted acids or Lewis acids such as AlCl₃, BF₃, and the like. Catalysts such as hydrotalcite or DMC may be also used when alkoxylates with a narrow distribution are desired.

Additionally, the alkoxylation may be carried out at temperatures in a range of about 80°-250° C., such as about 100°-220° C. The pressure may be between ambient pressure and 600 bar. If desired, the alkylene oxide may comprise a mixture of inert gas, for example approximately 5% to 60%.

As discussed above, the ethylene oxide, propylene oxide, butylene oxide units may be arranged within the inventive benzhydrylated alkoxylate compounds in any way. Thus, for example, the structural units a and b may be arranged at random or in blocks. The alkoxylation can therefore be carried out using only a single type of alkylene oxide, as well as a mixture of alkylene oxides. If, for example, a mixture of two or more different alkylene oxides is introduced into the reaction mixture, this generally leads, if the reactivity of the alkylene oxides is essentially comparable, to random polyether chains in which the constituents of the mixture are not present in any particular order. However, if the different alkylene oxides are fed into the reaction mixture in succession, i.e. a further alkylene oxide intended for the reaction is only fed in when that previously fed in has reacted completely, polyether segments are made up of blocks.

The degree of alkoxylation, i.e. the mean chain length of the polyether chains of aromatic alkoxylates according to the disclosure and their composition (in other words, the values of a and b) can be controlled by the ratio of the molar amounts of the benzhydrylated hydrophobe to alkylene oxide(s) employed in their preparation, and by the reaction conditions. In one embodiment, the amount of alkylene oxide used may range from about 0.5 to about 2 mole per mole of the benzhydrylated hydrophobe. Thus, on the one hand, the benzyhydrylated alkoxylates of the present disclosure may comprise at least or more than approximately 4, in some embodiments at least or more than approximately 10, in other particular embodiments at least or more than approximately 30, and still other embodiments at least or more than approximately 50 alkylene oxide units. On the other hand, the benzhydrylated alkoxylate compounds of the present disclosure may comprise not more than or less than approximately 100 or 70 or 40 or 10 alkylene oxide units.

The benzhydrylated alkoxylate compounds of the present disclosure may find use in a variety of applications, such as a surfactant for personal care and performance chemical formulations or as a reactant in the production of polyurea or polyurethane.

Thus, in one embodiment, there is provided a composition comprising a benzhydrylated alkoxylate compound comprising a compound having a formula

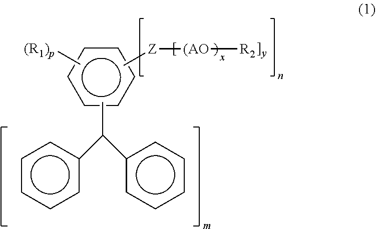

where: each R₁ is independently hydrogen, an alkyl group, a phenyl group, a group having a structure (2)

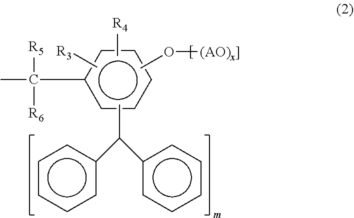

or two R₁'s in combination form a C₄H₄ di-radical having a structure (3)

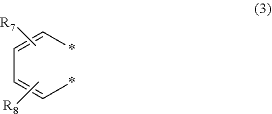

where * represents a binding site to the benzene ring;
each (AO) unit is independently an alkoxy group;
p is an integer of 1 to 4;
each R₂ is independently hydrogen, SO₃M, COOM or PO₃M₂;
M is hydrogen, a water-soluble cation, a monovalent metal or a polyvalent metal cation;
Z is sulfur, oxygen or nitrogen with the proviso that when Z is sulfur, y is 1 and n is 1 and when Z is oxygen, y is 1 and n is 1 or 2 and when Z is nitrogen, y is 2 and n is 1;
x is an integer of 1 to 100;
m is an integer of 1 to 3;
R₃ and R₄ are independently hydrogen, an alkyl group or a phenyl group;
R₅ and R₆ are independently hydrogen, methyl or a phenyl group; and
R₇ and R₈ are independently hydrogen or an alkyl group and at least one of a solvent, dispersant, or additive.

According to one embodiment the solvent is water, and in some embodiments, de-ionized water. In other embodiments a different solvent may be used in addition to or in place of water. Examples of such solvents include, but are not limited to, hydrocarbons (e.g. pentane or hexane), halocarbons (e.g. Freon 113), ethers (e.g. ethylether (Et₂O), tetrahydrofuran (“THF”) or diglyme (diethyleneglycol dimethyl ether)), nitriles (e.g. CH₃CN), or aromatic compounds (e.g. benzotrifluoride). Still further exemplary solvents include lactates, pyruvates, and diols. Solvents can also include, but are not limited to, acetone, 1,4-dioxane, 1,3-dioxolane, ethyl acetate, cyclohexanone, acetone, 1-methyl-2-pyrodidianone (NMP), and methyl ethyl ketone. Other solvents, include dimethylformamide, dimethylacetamide, N-methyl pyrrolidone, ethylene carbonate, propylene carbonate, glycerol and derivatives, naphthalene and substituted versions, acetic acid anhydride, propionic acid and propionic acid anhydride, dimethyl sulfone, benzophenone, diphenyl sulfone, phenol, m-cresol, dimethyl sulfoxide, diphenyl ether, terphenyl, and the like. Still further solvents include propylene glycol propyl ether (PGPE), 3-heptanol, 2-methyl-1-pentanol, 5-methyl-2-hexanol, 3-hexanol, 2-heptanol, 2-hexanol, 2,3-dimethyl-3-pentanol, propylene glycol methyl ether acetate (PGMEA), ethylene glycol, isopropyl alcohol (IPA), n-butyl ether, propylene glycol n-butyl ether (PGBE), 1-butoxy-2-propanol, 2-methyl-3-pentanol, 2-methoxyethyl acetate, 2-butoxyethanol, 2-ethoxyethyl acetoacetate, 1-pentanol, and propylene glycol methyl ether. The solvents enumerated above may be used alone or in combination.

In another embodiment, the composition may optionally contain a dispersant. In certain embodiments, the dispersant may be an ionic or a nonionic compound. The ionic or nonionic compound may further comprise a copolymer, an oligomer, or a surfactant other than the polyetheramine alkoxylate compound of formula (1), alone or in combination. The term copolymer, as used herein, relates to a polymer compound consisting of more than one polymeric compound such as block, star, dendrimer or grafted copolymers. Examples of a nonionic copolymer dispersant include polymeric compounds such as the tri-block EO-PO-EO co-polymers PLURONIC® L121, L123, L31, L81, L101 and P123 products. The term oligomer, as used herein, relates to a polymer compound consisting of only a few monomer units. Examples of ionic oligomer dispersants include SMA® 1440 and 2625 products.

The ionic or nonionic compound may also comprise a surfactant other than the benzhydrylated alkoxylate compound of formula (1) of the present disclosure. Surfactants useful in the composition of the present disclosure are well known and include anionic, nonionic, cationic and amphoteric compounds. Combinations of more than one such compounds may be used in the composition.

Anionic surfactant compounds which may be used include, but are not limited to, alkyl sulfates, alkyl benzene sulfonates, α-olefin sulfonates, alkyl taurates, alkyl sacrosinates, alkyl diphenyloxide disulfonates, alkyl naphthalene sulfonates, alkyl ether sulfates, alkyl ether sulfonates, sulfosuccinates, and other anionic surfactants as known for use in, for example, performance chemical formulations, including linear C₈₋₁₆ alkyl sulfates, C₈₋₁₆ alkyl sulfonates, C₈₋₁₆ alkyl benzene sulfonates and C₈₋₁₆ alkyl diphenyloxide disulfonates, decyl sulfophenoxy benzene/oxybis decyl benzene sulfonic acid disodium salt, and sodium octane sulfonate, sodium dodecyl sulfonate, sodium lauryl sulfate, and combinations of the foregoing. These surfactants are typically available as the alkali metal, alkaline earth and ammonium salts thereof.

Nonionic surfactant compounds which may be used include, but are not limited to, alkoxylates, N-substituted fatty acid amides, amine oxides, esters, sugar-based surfactants, polymeric surfactants, and mixtures thereof. Examples of alkoxylates are compounds such as non-aromatic alcohols, amines, amides, fatty acids or fatty acid esters which have been alkoxylated with 1 to 50 equivalents. Ethylene oxide and/or propylene oxide may be employed for the alkoxylation, usually ethylene oxide. Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters. Examples of polymeric surfactants are homo- or copolymers of vinylpyrrolidone, vinylalcohols, or vinylacetate.

Cationic surfactant compounds may also be used including quaternary surfactants, for example quaternary ammonium compounds with one or two hydrophobic groups, or salts of long-chain primary amines.

Amphoteric surfactant compounds which may be used include, but are not limited to, betaines, alkyl imidazolines, cocoamphopropionates, disodium cocoamphodipropionate (also known as cocoimidazoline carboxylate), or combinations thereof.

Other known additives, besides those described above, may optionally be added to the composition depending upon the application. These additives may include, but are not limited to, colorants, enzymes, wetting agents, antifoaming agents, buffering agents, pH adjusting agents, thickening agents, emulsifiers, anti-streaking agents, builders, chelating or sequestering agents, hydrotopes, anti-microbial agents, perfumes, herbicides, pesticides, fungicides, anti-oxidants, anti-wear additives, friction modifiers, viscosity index improvers, pour point depressants, corrosion inhibitors, solid carriers or fillers, protective colloids, adhesion agents, humectants, repellents, attractants, feeding stimulants, compatibilizers, bactericides, anti-freezing agents, crystallization inhibitors, tackifiers, binders, preservatives, clarifiers, fertilizers, UV stabilizers, salts, weighting agents, gravel particulates, gases, crosslinkers, thermodynamic hydrate inhibitors, kinetic hydrate inhibitors, clay stabilizing agents and mixtures thereof.

According to another embodiment, there is provided a composition comprising the benzhydrylated alkoxylate compound of formula (1) as described above and wherein the composition is substantially free of alkylphenol alkoxylates and mono-, di- and tristyrylphenol alkoxylates.

In yet another embodiment, there is provided a packaged product comprising: a) a container having at least an outlet; and b) a composition comprising the benzhydrylated alkoxylate compound of formula (1) as described above.

According to one embodiment, the packaged product of the present disclosure comprises a container having a closure means, such as a lid, cover, cap, or plug to seal the container. In another embodiment, the sealed container also has a nozzle or pour spout. The sealed container may have the shape of a cylinder, oval, round, rectangle, canister, tub, square or jug and contains the composition of the present disclosure.

The container may be made from any material, such as steel, glass, aluminum, cardboard, tin-plate, plastics including, but not limited to, high density polyethylene (HDPE), polypropylene (PP), polyvinyl chloride (PVC), polyethylene terephthalate (PET), oriented polypropylene (OPP), polyethylene (PE) or polyamide and including mixtures, laminates or other combinations of these.

In another embodiment, a concentrated composition comprising the benzhydrylated alkoxylate compound of formula (1) is provided that may be further diluted in water and/or other solvents to form an aqueous solution. A concentrated composition of the present disclosure, or “concentrate” allows one to dilute the concentrate to the desired strength and pH. A concentrate also permits longer shelf life and easier shipping and storage. Thus, in one embodiment there is provided a concentrate composition containing the benzhydrylated alkoxylate compound of the formula (1) of the present disclosure and water and/or other solvent and optionally a dispersant and/or one or more additives described above. For the concentrate, the amount of water (and in some embodiments, de-ionized water) and/or solvent may, for instance, be from about 0.5 to about 50% by weight, based on the total weight of the concentrate. Accordingly, the amount of the benzhydrylated alkoxylate compound of formula (1) (and optional dispersant and additives if present) contained in the concentrate may range from about 50% by weight up to 99.5% by weight, based on the total weight of the concentrate. As noted above, the concentrate may be further diluted with water, and in some embodiments, de-ionized water, and/or solvent to form the aqueous solution.

The composition including the benzhydrylated alkoxylate compound of formula (1) of the present disclosure described above may be used in a variety of applications and formulations, including, but not limited to, performance chemical formulations and personal care formulations.

Thus, in one embodiment there is provided a performance chemical formulation containing the composition comprising the benzhydrylated alkoxylate compound of formula (1) described above wherein the benzhydrylated alkoxylate compound of formula (1) is present in the performance chemical formulation in an amount ranging from about 0.01% by weight to about 40% by weight, based on the total weight of the performance chemical formulation. In another embodiment, there is provided a performance chemical formulation containing the composition comprising the benzhydrylated alkoxylate compound of formula (1) described above wherein the benzhydrylated alkoxylate compound of formula (1) is present in the performance chemical formulation in an amount ranging from about 0.1% by weight to about 30% by weight, based on the total weight of the performance chemical formulation. In still another embodiment, there is provided a performance chemical formulation containing the composition comprising the benzhydrylated alkoxylate compound of formula (1) described above wherein the benzhydrylated alkoxylate compound of formula (1) is present in the performance chemical formulation in an amount ranging from about 0.5% by weight to about 20% by weight, based on the total weight of the performance chemical formulation. In yet still another embodiment, there is provided a performance chemical formulation containing the composition comprising the benzhydrylated alkoxylate compound of formula (1) described above wherein the benzhydrylated alkoxylate compound of formula (1) is present in the performance chemical formulation in an amount ranging from about 1% by weight to about 10% by weight, based on the total weight of the performance chemical formulation.

Accordingly, in still another embodiment there is provided a personal care formulation containing the composition comprising the benzhydrylated alkoxylate compound of formula (1) described above wherein the benzhydrylated alkoxylate compound of formula (1) is present in the personal care formulation in an amount ranging from about 0.01% by weight to about 40% by weight, based on the total weight of the performance chemical formulation. In another embodiment, there is provided a performance chemical formulation containing the composition comprising the benzhydrylated alkoxylate compound of formula (1) described above wherein the benzhydrylated alkoxylate compound of formula (1) is present in the personal care formulation in an amount ranging from about 0.1% by weight to about 30% by weight, based on the total weight of the personal care formulation. In still another embodiment, there is provided a personal care formulation containing the composition comprising the benzhydrylated alkoxylate compound of formula (1) described above wherein the benzhydrylated alkoxylate compound of formula (1) is present in the personal care formulation in an amount ranging from about 0.5% by weight to about 20% by weight, based on the total weight of the personal care formulation. In yet still another embodiment, there is provided a personal care formulation containing the composition comprising the compound of formula (1) described above wherein the benzhydrylated alkoxylate compound of formula (1) is present in the personal care formulation in an amount ranging from about 1% by weight to about 10% by weight, based on the total weight of the personal care formulation.

In one particular embodiment, there is provided an agrochemical emuslfiable concentrate containing an agrochemical active component, a solvent and the benzhydrylated alkoxylate compound of formula (1).

Examples of agrochemical active components include, but are not limited to, a pesticide, fungicide, herbicide, insecticide, algicide, molluscicide, miticide, rodenticide, growth regulator or insect repellant. In one particular embodiment, the agrochemical active component includes an insecticide, such as Malathion, Chlorpyrifos, Cypermethrin and Chloropicrin, a herbicide, such as Trifluralin, 2,4-D Ester, MCPA Isooctylester, Metolachlor, Acetochlor, Triclopyr and Roundup®, or a fungicide, such as Mefenoxam and Etridiazole The agrochemical active component or components may be present in the agrochemical emulsifiable concentrate in an amount of at least about 5% w/w, or at least about 7.5% w/w, or at least about 10% w/w, where w/w means the weight of the agrochemical active component present in the agrochemical emulsifiable concentrate over the total weight of the agrochemical emulsifiable concentrate. In another embodiment, the agrochemical active component or components may be present in the agrochemical emulsifiable concentrate in an amount of less than about 80% w/w, or less than about 70% w/w, or less than about 60% w/w, or even less than about 50% w/w, where w/w means the weight of the agrochemical active component present in the agrochemical emulsifiable concentrate over the total weight of the agrochemical emulsifiable concentrate.

Examples of solvents include, but are not limited to, those described above. In another embodiment, the solvent may be a hydrocarbon, ether, phenol, glycol, lactone, chlorinated hydrocarbon, aromatic hydrocarbon nitrated hydrocarbon, dibasic ester, mono-ester such as ethyl acetate, butyl acetate, ethyl-3-ethoxy-propionate, propylene glycol methyl ether acetate, propylene glycol butyl ether acetate, dipropylene glycol methyl ether acetate, dipropylene glycol butyl ether acetate and a cyclic ester such as butyrolactone, organic sulfur-containing compounds dimethylsulfoxide (DMSO) and sulfolane, methyl ethyl ketone (MEK), 5-methyl-2-hexanone (MIAK), methyl isobutyl ketone and methyl isoamylbutone, a glycol ether such as propylene glycol methyl ether (PM), dipropylene glycol methyl ether (DPM), or dipropylene glycol n-butyl ether (DPNB), ethylene glycol butyl ether (EB) and dipropylene glycol butyl ether (DB), an alcohol such as methanol, ethanol, propanol, butanol, benzyl alcohol, an amide and mixtures thereof. In some embodiments, the solvent may be present in the agrochemical emulsifiable concentrate in an amount of at least 10% w/w, or at least about 15% w/w, or even at least about 20% w/w, where w/w means the weight of the solvent present in the agrochemical emulsifiable concentrate over the total weight of the agrochemical emulsifiable concentrate. In other embodiments, the solvent may be present in the agrochemical emulsifiable concentrate in an amount of less than about 80% w/w, or less than about 70% w/w or even less than about 60% w/w, where w/w means the weight of the solvent present in the agrochemical emulsifiable concentrate over the total weight of the agrochemical emulsifiable concentrate.

In another embodiment, the benzhydrylated alkoxylate compound of formula (1) may be present in the agrochemical emulsifiable concentrate in an amount of at least 1% w/w, or at least 5% w/w, or at least 7.5% w/w, where w/w means the weight of the benzhydrylated alkoxylate compound of formula (1) present in the agrochemical emulsifiable concentrate over the total weight of the agrochemical emulsifiable concentrate. In another embodiment, the benzhydrylated alkoxylate compound of formula (1) may be present in the agrochemical emulsifiable concentrate in an amount of less than about 20% w/w, or less than about 15% w/w, or less than about 10% w/w, where w/w means the weight of the benzhydrylated alkoxylate compound of formula (1) present in the agrochemical emulsifiable concentrate over the total weight of the agrochemical emulsifiable concentrate. In another embodiment, the benzhydrylated alkoxylate compound of formula (1) may be present in the agrochemical emulsifiable concentrate in an amount ranging from about 1% w/w to about 20% w/w, or from about 2% w/w to about 15% w/w, or from about 5% w/w to about 10% w/w, where w/w means the weight of the benzhydrylated alkoxylate compound of formula (1) present in the agrochemical emulsifiable concentrate over the total weight of the agrochemical emulsifiable concentrate.

The agrochemical emulsifiable concentrate may optionally comprise one or more additives described above in an amount up to about 20% w/w, where w/w means the weight of the additives present in the agrochemical emulsifiable concentrate over the total weight of the agrochemical emulsifiable concentrate. In one particular embodiment, the additives may be chosen from crystallization inhibitors, emulsifiers, surfactants other than the compound of formula (1), suspending agents, dyes, anti-oxidants, foaming agents, light absorbers, mixing aids, anti-foams, complexing agents, neutralizing or pH-modifying substances and buffers, corrosion-inhibitors, fragrances, wetting agents, absorption improvers, micronutrients, plasticizers, glidants, lubricants, dispersants, anti-freezes, and/or microbicides.

In another particular embodiment, there is provided an agrochemical suspension concentrate comprising an agrochemical active component, water and the benzhydrylated alkoxylate compound of formula (1).

In one embodiment, the agrochemical suspension concentrate may comprise at least about 1% w/w, or at least about 2% w/w, or even at least about 5% w/w, of the agrochemical active component or components, where w/w means the weight of the agrochemical active component or components present in the agrochemical suspension concentrate over the total weight of the agrochemical suspension concentrate. In another embodiment, the agrochemical suspension concentrate may comprise less than about 70% w/w, or less than about 60% w/w, less than about 50% by weight, or less than about 40% by weight of the agrochemical active component or components, where w/w means the weight of the agrochemical active component or components present in the agrochemical suspension concentrate over the total weight of the agrochemical suspension concentrate.

In another embodiment, the agrochemical suspension concentrate may contain at least about 10% w/w of water, or at least about 20% w/w, or even at least about 40% w/w, of water, where w/w means the weight of water present in the agrochemical suspension concentrate over the total weight of the agrochemical suspension concentrate. In another embodiment, the agricultural suspension concentrate may comprise less than about 90% w/w of water, or less than about 80% w/w, or even less than about 75% w/w of water, where w/w means the weight of water present in the agrochemical suspension concentrate over the total weight of the agrochemical suspension concentrate.

In yet another embodiment, the benzhydrylated alkoxylate compound of formula (1) may be present in the agrochemical suspension concentrate in an amount of at least 0.5% w/w, or at least 1% w/w, or at least 2.5% w/w, where w/w means the weight of the benzhydrylated alkoxylate compound of formula (1) present in the agrochemical suspension concentrate over the total weight of the agrochemical suspension concentrate. In another embodiment, the benzhydrylated alkoxylate compound of formula (1) may be present in the agrochemical suspension concentrate in an amount of less than about 10% w/w, or less than about 7.5% w/w, or less than about 5% w/w, where w/w means the weight of the benzhydrylated alkoxylate compound of formula (1) present in the agrochemical suspension concentrate over the total weight of the agrochemical suspension concentrate. In another embodiment, the benzhydrylated alkoxylate compound of formula (1) may be present in the agrochemical suspension concentrate in an amount ranging from about 0.01% w/w to about 10% w/w, or from about 0.75% w/w to about 7.5% w/w, or from about 1% w/w to about 5% w/w, where w/w means the weight of the benzhydrylated alkoxylate compound of formula (1) present in the agrochemical suspension concentrate over the total weight of the agrochemical suspension concentrate.

The agrochemical suspension may optionally contain one or more additives described above in an amount of up to 20% w/w, where w/w means the weight of the additives present in the agrochemical suspension concentrate over the total weight of the agrochemical suspension concentrate

In still further embodiments there is provided an agrochemical wettable powder” or “WP” which is intended to refer to a formulation comprising an agrochemical active ingredient in a finely ground state combined with the benzhydrylated alkoxylate compound of formula (1) and optionally additives described above and bulking agents. The benzhydrylated alkoxylate of the present disclosure may be used in any of the amounts described above to facilitate the dispersion of wettable powders into a fluid medium, for e.g. water.

In still yet another embodiment, there is provided an agrochemical “water-dispersible granule” which is intended to refer to an agrochemical active ingredient-containing formulation that is in a granular phase and may be used after being diluted with a fluid medium, for e.g. water. The benzhydrylated alkoxylate of the present disclosure may be added at any of the amounts described above to facilitate the dispersion of water-dispersible granules into the fluid medium.

### EXAMPLES

### Example 1. Synthesis of tribenzyhydryl phenol ethoxylate

The following generally describes a process to prepare a benzhydryl phenol ethoxylate compound according to the present disclosure:

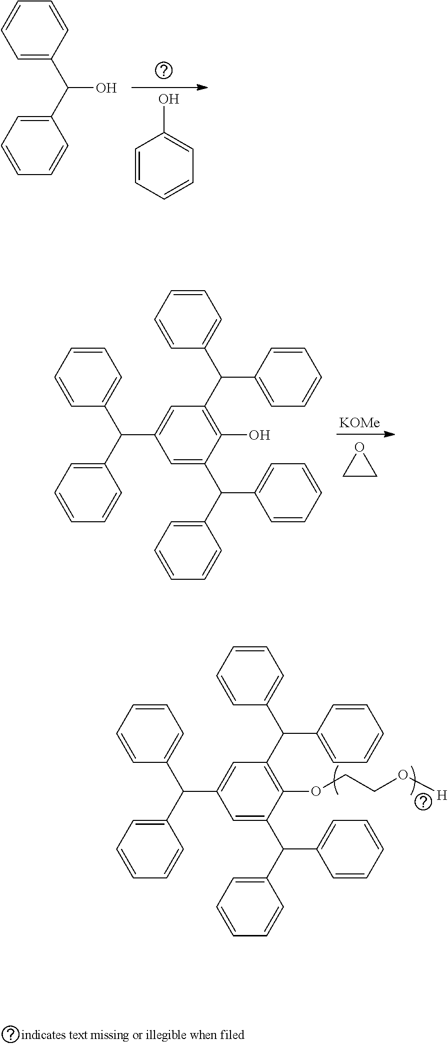

200 grams of phenol and 1173 grams of benzhydrol were melted and combined in a reactor. 1.3 grams of para-toluenesulfonic acid was then added at a temperature of 80° C. and the reaction was further heated to 170° C. and held for 5 hours while stripping out water. The resulting tri-benzhydryl phenol hydrophobe, an orange, glassy solid, was then added to a high-pressure kettle and charged with a catalytic amount of potassium methoxide, stripped of water and methanol, and heated to 120° C. The appropriate amount of ethylene oxide was metered in at a rate that kept the reactor temperature between 145° C. and 150° C. and the pressure below 70 psig. After the ethylene oxide addition was complete, the reaction was digested for 2 hours and then stripped of any residual ethylene oxide. Several inventive compounds were produced in this manner that contained 15, 20, 25, or 30 moles ethylene oxy units.

### Example 2. Dibenzhydryl Resorcinol Ethoxylate

200 grams of resorcinol and 670 grams of benzhydrol were melted and combined in a reactor. 1.0 gram of para-toluenesulfonic acid was then added at a temperature of 80° C. and the reaction was further heated to 170° C. and held for 5 hours while stripping out water. The resulting di-benzhydryl resorcinol hydrophobe, an orange, glassy solid, was then added to a high-pressure kettle and charged with a catalytic amount of potassium methoxide, stripped of water and methanol, and heated to 120° C. An appropriate amount of ethylene oxide was metered in at a rate that kept the reactor temperature between 145° C. and 150° C. After the ethylene oxide addition was complete, the reaction was digested for 2 hours and then stripped of any residual ethylene oxide. Several inventive compounds were produced in this manner that contained 15, 20, 25, or 30 moles ethylene oxy units.

### Example 3. Dibenzhydryl Nonylphenol Ethoxylate

The following generally describes a process to prepare an alkyl substituted benzhydryl phenol ethoxylate compound according to the present disclosure:

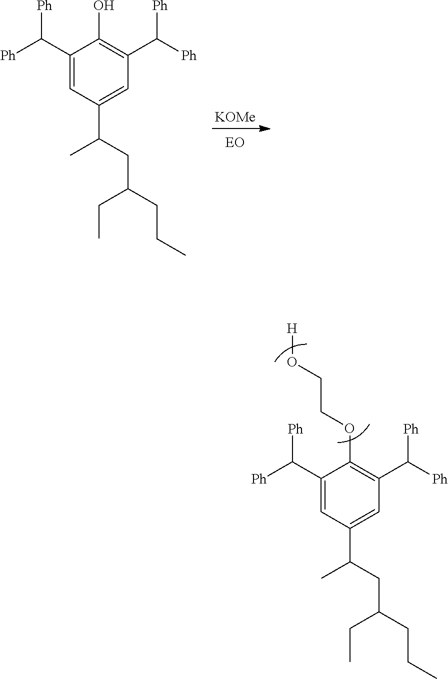

200 grams of nonylphenol and 334 grams of benzhydrol were melted and combined in a reactor. 0.5 grams of para-toluenesulfonic acid was then added when the reaction reached 80° C. and the reaction was further heated at 170° C. for 5 hours while stripping out water. The resulting Dibenzhydryl nonylphenol, an amorphous solid, was then was then added to a high-pressure kettle and charged with a catalytic amount of potassium methoxide, stripped of water and methanol, and heated to 120° C. An appropriate amount of ethylene oxide was metered in at a rate that kept the reactor temperature between 145° and 150° C. After the ethylene oxide addition was complete, the reaction was digested for 2 hours and then stripped of any residual ethylene oxide. Several inventive compounds were produced in this manner that contained 15, 20, 25, or 30 moles ethylene oxy units per hydrophobe.

### Example 4. Tetrabenzhydryl Bisphenol a Hydrophobe

300 grams of bisphenol A were added to a flask and heated, and 968 grams benzhydrol were added and melted in the reactor. 1.3 grams of para-toluenesulfonic acid was then added to the mixture at 80° C. and the reaction was heated to 170° C. while under a nitrogen atmosphere. Water of reaction was removed for 5 hours, after which the reaction was complete. This hydrophobe may be converted to an alkoxylate as described above.

### Example 6. Tribenzhydryl Aniline Hydrophobe

200 grams of aniline and 1187 grams of benzhydrol were added to a round bottom flask equipped with an overhead stirrer, and heated to 70° C. to a melt. 2.0 grams of para-toluenesulfonic acid was then added and the mixture was heated to 180° C. for 5 hours under a nitrogen atmosphere. The reaction was complete when water evolution ceased. This hydrophobe may be converted to an alkoxylate as described above.

### Example 7. Characterization

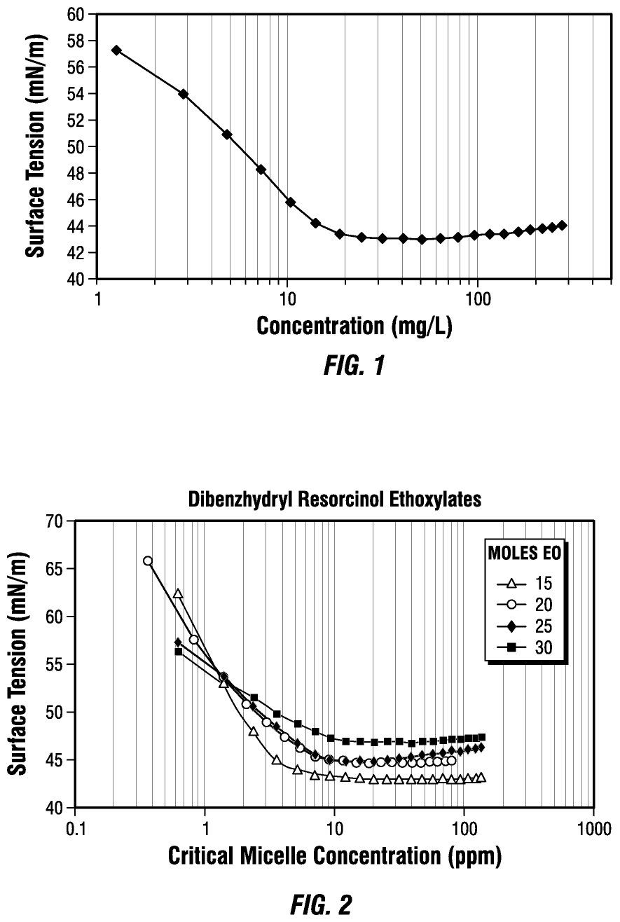

Tribenzyhydryl phenol ethoxylate (20 moles ethylene oxy units) produced in Example 1 was characterized using a surface tensiometer to determine its critical micelle concentration and surface tension. FIG. 1 shows that this compound has surface properties that are typical for conventional nonionic surfactants. The critical micelle concentration for this compound was determined to be 16.5 mg/L.

Dibenzhydryl resorcinol ethoxylates produced in Example 2 were characterized using a surface tensiometer to determine critical micelle concentration and surface tension. FIG. 2 shows that these compounds have exceptionally low critical micelle concentrations for nonionic surfactants, for example, the compound having 15 moles ethylene oxy units has a critical micelle concentration of 4 ppm.

While the foregoing is directed to various embodiment s of the present disclosure, other and further embodiments of the disclosure may be devised without departing from the basic scope thereof, and the scope thereof is determined by the claims that follow.

1. A benzyhydrylated alkoxylate comprising a compound having a formula (1) 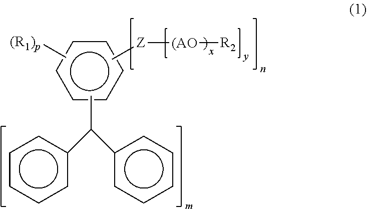
where: each R₁ is independently hydrogen, an alkyl group, a phenyl group, a group having a structure (2) 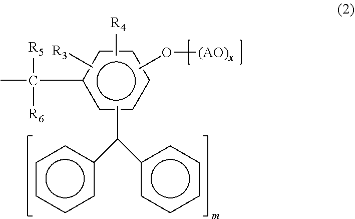
or two R₁'s in combination form a C₄H₄ di-radical having a structure (3) 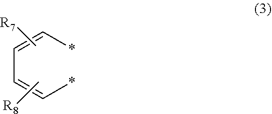
where * represents a binding site to the benzene ring;
each (AO) unit is independently an alkoxy group;
p is an integer of 1 to 4;
each R₂ is independently hydrogen, SO₃M, COOM or PO₃M₂;
M is hydrogen, a water-soluble cation, a monovalent metal or a polyvalent metal cation;
Z is sulfur, oxygen or nitrogen with the proviso that when Z is sulfur, y is 1 and n is 1 and when Z is oxygen, y is 1 and n is 1 or 2 and when Z is nitrogen, y is 2 and n is 1;
x is an integer of 1 to 100;
m is an integer of 1 to 3;
R₃ and R₄ are independently hydrogen, an alkyl group or a phenyl group;
R₅ and R₆ are independently hydrogen, methyl or a phenyl group; and
R₇ and R₈ are independently hydrogen or an alkyl group.

2. The benzyhydrylated alkoxylate of claim 1, wherein Z is oxygen and n is 1.

3. The benzyhydrylated alkoxylate of claim 1, wherein Z is oxygen and n is 2.

4. The benzhydrylated alkoxylate of claim 1, wherein Z is nitrogen.

5. The benzhydrylated alkoxylate of claim 1, wherein each R₂ is hydrogen.

6. The benzhydrylated alkoxylate of claim 1, wherein each R₁ is hydrogen.

7. The benzhydrylated alkoxylate of claim 1, wherein one R₁ is the group having the structure 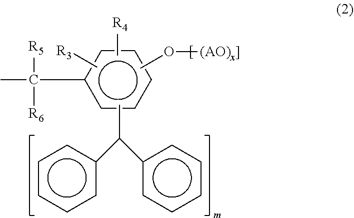
where AO, x, m, R₃, R₄, R₅ and R₆ are defined as above.

8. The benzhydrylated alkoxylate of claim 1, wherein two R₁'s in combination form the C₄H₄ di-radical having a structure 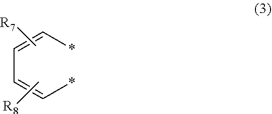
where * represents a binding site to the benzene ring; and R₇ and R₈ are independently hydrogen or an alkyl group.

9. A method for preparing the benzyhydrylated alkoxylate of claim 1 comprising the steps of: reacting a benzhydrol with an aromatic compound selected from phenol optionally substituted with an alkyl group, a phenyl group or combination thereof, a bisphenol, a benzenediol optionally substituted with an alkyl group, naphthol, thiophenol and aniline optionally substituted with an alkyl group in the presence of an acid catalyst to form a benzhydrylated hydrophobe; and, alkoxylating the benzyhydrylated hydrophobe with an alkylene oxide to form the benzhydrylated alkoxylate.

10. The method of claim 9, wherein the method further comprises the step of reacting the benzhydrylated alkoxylate with an acidic moiety and optionally neutralizing with a source of alkali metal, alkaline earth metal, amine or ammonia.

11. A composition comprising the benzyhydrylated alkoxylate of claim 1 and a solvent.

12. A packaged product comprising: a) a container having at least an outlet; and b) the composition of claim 11.

13. A concentrate composition comprising from about 50% by weight to about 99.5% by weight of the benzhydrylated alkoxylate of claim 1 and from about 0.5% by weight to about 50% by weight of water and optionally one or more additives, where the % by weight is based on the total weight of the concentrate composition.

14. A performance chemical formulation or personal care formulation comprising the composition of claim 11, wherein the benzhydrylated alkoxylate is present in an amount ranging from about 0.01% by weight to about 40% by weight, based on the total weight of the performance chemical formulation.

15. A personal care formulation comprising the composition of claim 11, wherein the benzhydrylated alkoxylate is present in an amount ranging from about 0.01% by weight to about 40% by weight, based on the total weight of the personal care formulation.

16. An agrochemical emulsifiable concentrate comprising an agrochemical active component, a solvent and the composition of claim 11 wherein the benzhydrylated alkoxylate is present in an amount ranging from about 1% w/w to about 20% w/w, where w/w means the weight of the benzhydrylated alkoxylate present in the agrochemical emulsifiable concentrate over the total weight of the agrochemical emulsifiable concentrate.

17. An agrochemical suspension concentrate comprising an agrochemical active component, water and the composition of claim 11 wherein the benzhydrylated alkoxylate is present in the agrochemical suspension concentrate in an amount ranging from about 0.01% w/w to about 10% w/w, where w/w means the weight of the benzhydrylated alkoxylate present in the agrochemical suspension concentrate over the total weight of the agrochemical suspension concentrate.

18. An agrochemical soluble liquid concentrate comprising a dissolved agrochemical active ingredient, water, and the composition of claim 11 wherein the benzhydrylated alkoxylate is present in the agrochemical soluble liquid concentrate in an amount ranging from about 0.01% to about 20% w/w, where w/w means the weight of the benzhydrylated alkoxylate present in the agrochemical soluble liquid concentrate over the total weight of the agrochemical soluble liquid concentrate.

19. An agrochemical wettable powder comprising an agrochemical active ingredient in a finely ground state, the benzhydrylated alkoxylate of claim 1 and optionally an additive.
